(19) United States
(12) Patent Application Publication
(10) Pub. No.: US 2012/0197239 A1
(43) Pub. Date: Aug. 2, 2012

(54) ENDOSCOPIC MEDICAL DEVICE WITH ARTICULATING JOINTS

(76) Inventors: Paul Smith, Smithfield, RI (US); Gary Kappel, Acton, MA (US); John Golden, Norton, MA (US); Barry Weitzner, Acton, MA (US)

(21) Appl. No.: 13/338,391

(22) Filed: Dec. 28, 2011

Related U.S. Application Data
(60) Provisional application No. 61/437,996, filed on Jan. 31, 2011.

(51) Int. Cl. A61B 17/00

(52) U.S. Cl. 606/1

(57) ABSTRACT

An endoscopic device may include an elongate section extending from a proximal end to a distal end along a longitudinal axis. The elongate section may be configured to transmit a desired torque about the longitudinal axis without relative rotation between the proximal end and the distal end. The device may also include a plurality of bend elements positioned on the elongate section. The plurality of bend elements may include a first articulating joint configured to bend the elongate section in a first bending plane, and a second articulating joint configured to bend the elongate section in a second bending plane different from the first bending plane.

### CROSS-REFERENCE TO RELATED APPLICATIONS

This application is based upon and claims the benefit of priority under 35 U.S.C §119(e) of U.S. Provisional Application No. 61/437,996 to Smith et al. filed on Jan. 31, 2011.

### DESCRIPTION OF THE INVENTION

1. Field of the Invention

Embodiments of the present invention relate to medical devices used in endoscopic applications. In particular, exemplary embodiments of the present invention relate to endoscopic medical devices with articulating joints that provide for torque transmission and at least one bending degree of freedom.

2. Background of the Invention

An endoscopic device, such as, for example, a catheter, is an instrument introduced into the body for diagnostic or therapeutic purposes. For example, for injecting a drug at the site within the body, an injection catheter is used. Such a catheter includes a needle at the distal end of a sheath through which the drug is directed to the needle. An outer sheath over the injection sheath may also be provided as a passageway for irrigation fluid around the needle and/or as a protective encasement for the needle as the distal end of the catheter is moved to the therapy site. Endoscopic catheters equipped for other applications, e.g., delivery of a surgical tool to an internal body site, similarly generally include a mechanical feature at the distal end of catheter that may be encased in an outer sheath.

Many endoscopic therapeutic procedures are directed to deep internal regions in the body that require an appropriately equipped catheter to be of substantial length to reach the site of interest within the body. To maneuver through tortuous body tracts without causing trauma to the walls of the tract, the catheter also requires flexibility and the ability to conform to curves in the body tract as the catheter is advanced to the intended internal site. Since the catheter is also used to direct a force, such as a torsional or a twisting force, to a surgical tool coupled to the distal end of the catheter, these catheters must further be capable to transmitting the torsional force without undergoing relative displacement or relative rotation between the proximal and distal ends of the catheter.

A typical endoscopic catheter may have limited ability to bend while transmitting torque. For instance, some known endoscopic catheters include a sheath made of one or more helically wound strips that impart flexibility to the endoscope. However, this flexible sheath may limit the amount of torque that may be transmitted to the distal end due to relative rotation between proximal and distal sections of the catheter. Effective therapeutic procedures may require that surgical tools delivered to an internal site possess the ability to transmit torque and bend in multiple planes while transmitting the torque. The limited freedom of movement of conventional endoscopic catheters may hinder these tasks. In some cases, the endoscopic catheter with a surgical tool at its distal end may be delivered through a working channel of an endoscope. In this case, a distal end of the endoscope may be inserted into a body tract and pushed into the tract until the distal end is appropriately positioned at or near the internal site. The catheter may then be delivered to the internal site through a working channel of the endoscope. In this case, both the endoscope and the catheter will need the ability to flex and conform with curves in the body tract.

Embodiments of the disclosed endoscopic catheters with articulating joints are directed to overcoming these and/or shortcomings in existing technology. The current disclosure is broadly applicable to any catheter or other device that is delivered to an internal site within the body through a body tract. However, for the sake of brevity, aspects of the current disclosure will be described with reference to an endoscopic catheter.

### SUMMARY OF THE INVENTION

An embodiment of the invention may include an endoscopic device. The endoscopic device may include an elongate section extending from a proximal end to a distal end along a longitudinal axis. The elongate section may be configured to transmit a desired torque about the longitudinal axis without relative rotation between the proximal end and the distal end. The device may also include a plurality of bend elements positioned on the elongate section. The plurality of bend elements may include a first articulating joint configured to bend the elongate section in a first bending plane, and a second articulating joint configured to bend the elongate section in a second bending plane different from the first bending plane.

Various embodiments of the invention may also include an endoscopic device wherein the first bending plane is orthogonal to the second bending plane; wherein at least one of the first articulating joint and the second articulating joint includes a live hinge; wherein the first articulating joint includes a stop mechanism that is configured to limit the amount of deflection of the first articulating joint; wherein at least one of the first articulating joint and the second articulating joint includes a rectangular strip that is folded along a width of the strip; wherein the first articulating joint and the second articulating jointure substantially similar parts that are oriented such that the first articulating joint bends in the first bending plane and the second articulating joint bends in the second bending plane; wherein at least one of the first articulating joint and the second articulating joint includes one of a stainless steel, spring steel, a shape memory alloy, and a plastic material; wherein the bend elements further include a third articulating joint that is configured to bend the elongate section in a third bending plane that is different from the first and second bending planes; wherein the device further includes a string of first articulating joints in one section of the elongate section and a string of second articulating joints in a different section of the elongate section; wherein the device further includes a first elongate member that is coupled to the first articulating joint and configured to bend the first articulating joint in the first bending plane; and wherein the device further includes a second elongate member that is coupled to the second articulating joint and configured to bend the second articulating joint in the second bending plane.

Another embodiment of the invention may include an endoscopic device that includes an elongate section extending from a proximal end to a distal end along a longitudinal axis. The elongate section may be configured to transmit a desired torque about the longitudinal axis without relative rotation between the proximal end and the distal end. The device may include a lumen extending through the elongate section from the proximal end to the distal end, and a plurality of first bend elements positioned along the elongate section and a plurality of second bend elements positioned along the elongate section. Each bend element of the plurality of first bend elements may be configured to bend the elongate section along a first bending plane, and each bend element of the plurality of second bend elements may be configured to bend the elongate section along a second bending plane different from the first bending plane.

Various embodiments of the invention may also include an endoscopic device wherein the first bending plane is orthogonal to the second bending plane; wherein the plurality of first bend elements includes multiple first bend elements positioned proximate each other in a first length of the elongate section, and the plurality of second bend elements include multiple second bend elements positioned proximate each other in a second length of the elongate section, the first length being different from the second length; wherein the plurality of first bend elements includes a strip of material that is folded to form multiple first bend elements that are each configured to bend in the first bending plane; wherein the plurality of second bend elements includes multiple second bend elements that are folded from the strip of material and are each configured to bend in the second bending plane; and wherein at least one of the plurality of first bend elements and the plurality of second bend elements includes a pivot.

Another embodiment of the invention may include a method of using an endoscopic catheter. The method may include inserting a distal end of the catheter into an opening in ct of a body. The catheter may include an elongate section extending along a longitudinal axis and a plurality of bend elements. The elongate section may be configured to transmit a torque about the longitudinal axis without relative rotation between regions of the elongate section on either side of the plurality of bend elements. The plurality of bend elements may include at least a first and a second articulating joint. The first and the second articulating joints may be configured to bend the elongate section in different bending planes. The method may also include activating the first articulating joint to bend the first articulating joint in a first bending plane, and activating the second articulating joint to bend the second articulating joint in the second bending plane.

Various embodiments of the invention may also include a method wherein activating the first articulating joint includes bending the first articulating joint in the first bending plane without bending the second articulating joint; and the method may further include applying a torsional force at a proximal end of the catheter to transmit the force to a distal end of the catheter without causing relative rotation between the proximal and distal ends of the catheter.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 is a schematic view of an exemplary embodiment of an endoscopic catheter of the current disclosure.

FIGS. 2A and 2B are illustrations of an embodiment of articulating joints in a length of the catheter of FIG. 1.

FIGS. 3A and 3B are illustrations of another embodiment of articulating joints in a length of the catheter of FIG. 1.

FIG. 4 is an illustration of another embodiment of articulating joints in a length of the catheter of FIG. 1.

FIGS. 5A and 5B are illustrations of an embodiment of an articulating joint in the catheter of FIG. 1.

FIG. 5C is an illustration of another embodiment of an articulating joint in the catheter of FIG. 1.

FIGS. 6A and 6B are illustrations of another embodiment of articulating joints in a length of the catheter of FIG. 1.

FIGS. 7A and 7B are illustrations of another embodiment of articulating joints in a length of the catheter of FIG. 1.

FIG. 8 is an illustration of another embodiment of articulating joints in a length of the catheter of FIG. 1.

### DESCRIPTION OF THE EMBODIMENTS

Reference will now be made to exemplary embodiments of the invention, examples of which are illustrated in the accompanying drawings. Wherever possible, the same reference numbers will be used throughout the drawings to refer to the same or like parts.

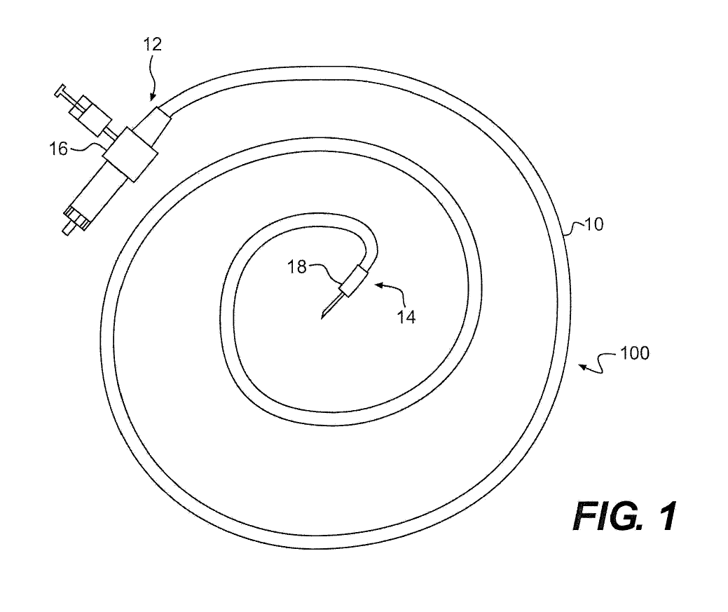

FIG. 1 depicts an exemplary catheter 100 in accordance with the current disclosure. Catheter 100 includes a cannula or a tubular catheter body 10 that extends from a proximal end 12 to a distal end 14. An endoscopic instrument 18, that is configured to assist in a desired endoscopic procedure within the body, may be coupled to the distal end 14 of the catheter 100. The distal end 14, along with endoscopic instrument 18, may be configured for insertion into a body tract through a natural anatomic opening or an incision on the body. Tubular body 10 may include a substantially circular cross-sectional shape with a uniform outer diameter. However, this configuration is only exemplary, and other embodiments of catheter 100 may have a different cross-sectional shape and/or a non-uniform diameter. Tubular body 10 may be flexible and include a length that is sufficient for the desired endoscopic procedure. Tubular body 10 may be made of one or more biocompatible or other materials that are known in the art. In some embodiments, the external surface of tubular body 10 may include a lubricious coating to assist in the insertion of the catheter 100 into the body.

One or more lumens may extend lengthwise through the catheter 100 from the proximal end 12 to the distal end 14. The lumens may extend through the tubular body 10 and may have any cross-sectional shape and size. The lumens may serve as conduits for supplying additional surgical tools, devices, and/or fluids (such as, for example, a gas) to the work site. These devices may include, among others, devices to illuminate the work site, devices to image the work site, and devices that deliver supplies such as fluid, suction, etc. to the work site. The proximal end 12 of catheter 100, that remains outside the body, may be coupled to an activation mechanism 16 that may be configured to control the catheter 100 and the devices at the distal end 14, such as, for example, the endoscopic instrument 18. Control of the endoscopic instrument 18 may include operating the endoscopic instrument 14 to perform a desired diagnostic or therapeutic procedure at the work site.

Control of the catheter 100 may include bending the tubular body 12 of the catheter 100 in a controlled manner to navigate the catheter 100 through a tortuous body tract as it traverses from the point of insertion into the body to the work site within the body. Bending of the tubular body 10 may be achieved by one or more elongate members extending through the tubular body 10 from the proximal end 12 to the distal end 14 thereof. These elongate members may include any member (such as, for example, strings, rods, links, etc.) that is configured to transmit a tensile and/or a compressive force from the proximal end to the distal end. In an embodiment of a catheter 100 which is configured to bend in one plane, two elongate members may be positioned at diametrically opposite ends of the catheter 100 in the desired bending plane. These elongate members may be attached at the distal end 14 to the catheter 100 and to a knob or another control mechanism of the activation mechanism 16 at the proximal end 12. The catheter 100 may be controllably bent in the bending plane by pulling or pushing these elongate members in a coordinated manner. Tubular body 10 may include articulating joints that are configured to controllably bend in response to activation by the activation mechanism 16. A protective sheath or covering may cover these articulating joints and extend along the outside of the tubular body 10 to protect the inside of the catheter 100 from bodily fluids. In some embodiments, the sheath may include a suitable coating (such as, for example, a lubricious coating) to facilitate insertion through an endoscopic channel.

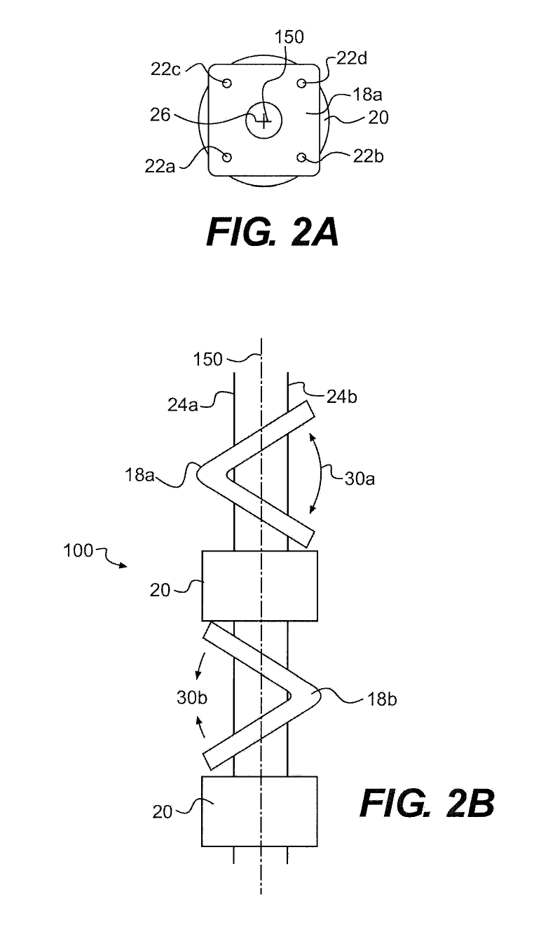

FIGS. 2A and 2B illustrate a portion of the bendable tubular body 10 with its protective sheath removed to show an embodiment of the articulating joints therein. FIG. 2A illustrates a side view and FIG. 2B illustrates a top view of the portion of the tubular body. In the description below, reference may be made to both FIGS. 2A and 2B. Although FIG. 2B only depicts two articulating joints, catheter 100 may include a plurality of articulating joints aligned or stacked along a longitudinal axis 150. These articulating joints may include v-shaped parts that are adapted enable the tubular body 10 to bend in a controlled manner in response to activation by activation mechanism 16. These stacked articulating joints may include a first articulating joint 18a and a second articulating joint 18b coupled to each other through a tubular section 20. In some embodiments of catheter 100, this pattern of articulating joints (a first articulating joint 18a and a second articulating joint 18b with a tubular section 20 in between) may be replicated along substantially the entire length of tubular body 10, while in other embodiments this pattern of articulating joints may only be incorporated in selected regions of tubular body 10. Although a particular pattern of parts (first articulating joint 18a, second articulating joint 18b, and tubular section 20) is illustrated in FIGS. 2A and 2B, this is only exemplary. In general, any suitable combination of these parts may be used along the length of tubular body 10.

First articulating joint 18a and second articulating joint 18b may include through-holes 22a-22d that are adapted to pass elongate members 24a-24d therethrough (24c and 24d are not visible in FIG. 2B). The number of through-holes may depend upon the application, and in general, any number of through-holes may be provided. In the embodiment illustrated in FIGS. 2A and 2B, four through-holes are provided through first articulating joint 18a and second articulating joint 18b. These through-holes may be arranged in a manner such that, pulling and/or pushing elongate members 24a-24d may controllably bend the articulating joints in the desired directions. In some embodiments, these through-holes 24a-24d may be arranged on diametrically opposite ends of first articulating joint 18a and second articulating joint 18b. Under such an arrangement, pulling elongate members 24b and 24d and/or pushing elongate members 24a and 24c may cause the legs of first articulating joint 18a to traverse in the direction of the arrow labeled 30a, and the legs of second articulating joint 18b to traverse in the direction of the arrow labeled 30b. Such a movement of the legs may bend the top portion of the catheter 100 towards the left in FIG. 2B (that is, in a counterclockwise direction).

Similarly, pulling elongate members 24a and 24c and/or pushing elongate members 24b and 24d may cause the legs of the first and second articulating joints 18a, 18b to move in the opposite direction, to result in the top of the catheter 100 to bend in the clockwise direction. The actuation mechanism 16 may thus push/pull the elongate members 24a-24d to bend tubular body 10 to maneuver the catheter 100 through a curved body tract as the catheter 100 passes therethrough. The actuation mechanism 16 may also include other features to assist with the maneuvering of the catheter 100 through a body tract. For example, in some embodiments, the actuating mechanism 16 may include a locking mechanism to keep the tubular body 10 in a fixed bend configuration.

The articulating joints 18a and 18b are also configured to enable the tubular body 10 to transmit torque about the longitudinal axis 150 without rotation of a length of the catheter 100 on one side of the articulating joints relative to a length of the catheter 100 on the opposite side of the articulating joints. In an exemplary application of catheter 100, it may be desirable to transmit a torque to a device coupled to the distal end of the catheter 100 to assist in a medical procedure. Relative rotation (about longitudinal axis 150) between different lengths of the catheter 100 may decrease the amount of torsional force that may be transmitted from the proximal end to the distal end. Since a rotation of the catheter 100 about the longitudinal axis 150 does not activate articulating joints 18a and 18b, relative rotation between sections of the catheter 100 on either side of these joints does not occur. Therefore, torque transmission of the catheter 150 about the longitudinal axis 150 is unaffected by the articulating joints of the current disclosure.

It should be noted that the term “elongate member” is used in this disclosure to broadly refer to any link, flexible or rigid, that is configured to activate the articulating joints. It should also be noted that, although four through-holes 22a-22d and four elongate members 24a-24b are illustrated in FIGS. 2A and 2B, this is only exemplary and other embodiments of the catheter may have a different arrangement (such as, for example, a different number of through-holes and elongate members). For instance, in some embodiments, only two elongate members may be provided to bend the catheter 100 in both clockwise and counterclockwise directions in a bending plane. In such an embodiment, first articulating joint 18a and second articulating joint 18b may only be provided with only two through-holes positioned on diametrically opposite ends of the bending plane. The two through-holes may each include a different one of a first elongate member and a second elongate member passing therethrough. Pulling the first elongate member (and/or pushing the second elongate member) may bend catheter 100 in one direction, and pulling the second elongate member (and/or pushing the first elongate member) may bend the catheter in the opposite direction.

The first articulating joint 18a and second articulating joint 18b may also include a cavity 26 that extends through the catheter 100 along the longitudinal axis 150. Although a circular cavity 26 is depicted to be centrally positioned on first articulating joint 18a and second articulating joint 18b, this is only exemplary. In general, cavity 26 may have any shape and may be positioned at any location. In some embodiments, cavity 26 may also include a coating (such as, for example, a lubricious coating). Cavity 26 may extend through substantially the entire length of the tubular body 10, and may be used to deliver a surgical tool (or another device) from proximal end 12 to distal end 14 of catheter 100.

Tubular section 20 positioned between the first articulating joint 18a and the second articulating joint 18b may also include through-holes and a cavity that are positioned to align with the through-holes and the cavity 26 of the first and second articulating joints 18a, 18b. Elongate members 24a-24d may extend through these through-holes on the tubular section 20 to couple the first articulating joint 18a and the second articulating joint 18b together. These elongate members may extend along substantially an entire length of the tubular body 10 to couple the multiple articulating joints along the length of catheter 100. Although the tubular section 20 illustrated in FIGS. 2A and 2B has a circular cross-sectional shape, in general, tubular section 20 may have any shape and configuration.

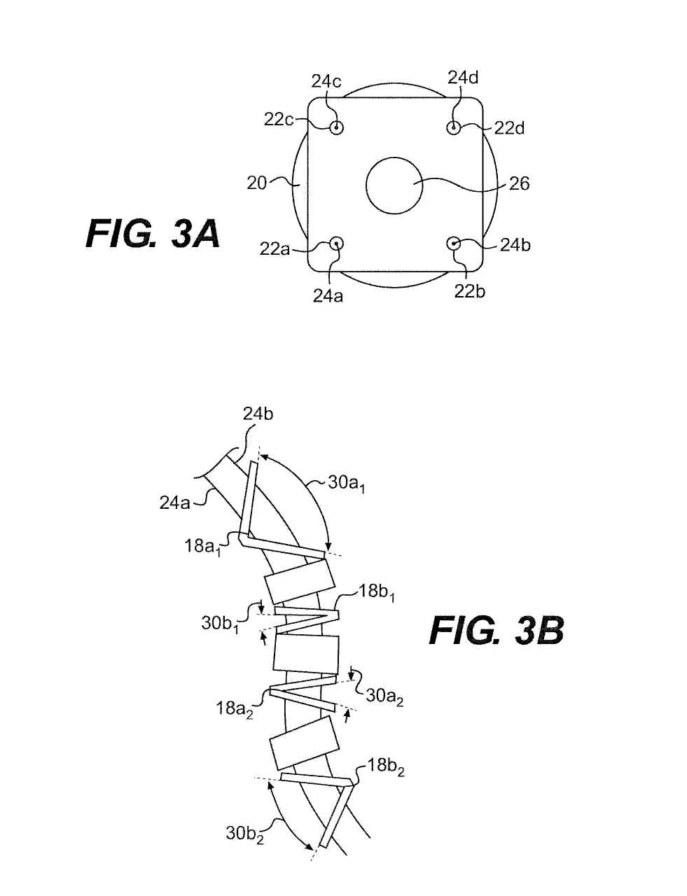

In some embodiments multiple articulating joints on the catheter 100 may be controlled independently. In these embodiments, a pair of opposite elongate members may be coupled to a first set of articulating joints and another pair of opposite elongate members may be coupled to a second set of articulating joints. In these embodiments, one pair of elongate members may be pulled/pushed to bend the first set of articulating joints and the other pair of elongate members may be pulled/pushed to bend the second set of articulating joints. One such embodiment is illustrated in FIGS. 3A and 3B. FIG. 3A illustrates a top view and FIG. 3B illustrates a side view of this embodiment. In the embodiment of FIGS. 3A and 3B, a first pair of opposite elongate members 24a and 24b may be coupled to a first set of articulating joints (18a₁ and 18b₁) and another pair of opposite elongate members 24c, 24d may be coupled to a second set of articulating joints (18a₂ and 18b₂). Pulling elongate member 24b (and/or pushing elongate member 24a) may cause the legs of first set of articulating joints to move in the directions of arrows 30a₁ and 30b₁ respectively. Elongate member 24d and elongate member 24c may be independently activated to bend the second set of articulating joints in the same direction as, or in the opposite direction of, the first set of articulating joints. For instance, elongate member 24d may be pulled (and/or elongate member 24c may be pushed) to bend the legs of the second set of articulating joints (18a₂ and 18b₂) in the same direction as the legs of the first set of articulating joints, and elongate member 24c may be pulled (and/or elongate member 24d may be pushed) to bend these legs in the opposite direction. Activating the multiple sets of articulating joints of catheter 100 in an independent manner may enable different lengths of the catheter 100 to bend in different directions. Such a capability may assist catheter 100 in traversing body tracts that bend in different directions with minimal trauma to the walls of the body tract.

Although in FIGS. 2A-2B and 3A-3B, a set of articulating joints is shown to include two joints, this is only exemplary and other embodiments may include a different number and/or arrangement of articulating joints. For instance, in some embodiments, a set of articulating joints may include only one joint, while in another embodiments several articulating joints (such as, for example, six articulating joints) may make up a set of joints. The desired bending characteristics of the catheter 100 (such as, for example, bending radius, etc.) may dictate the structure and number of articulating joints.

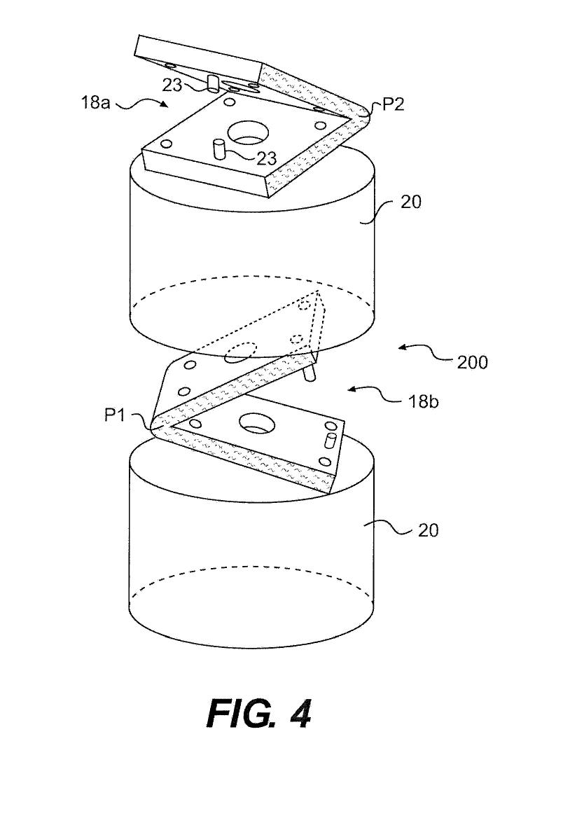

In the embodiments of the catheter 100 illustrated in FIGS. 2A-2B and 3A-3B, each articulating joint is oriented to enable the catheter 100 to bend in a single bending plane (the plane of the paper in FIGS. 2B and 3B). In some embodiments, the joints may be arranged to enable the catheter 100 to bend in multiple bending planes. FIG. 4 illustrates an arrangement of articulating joints that is configured to enable catheter 200 to bend along two different bending planes P1 and P2 in response to activation. The articulating joints in the embodiment of FIG. 4 include a first articulating joint 18a and a second articulating joint 18b that are oriented such that the first articulating joint 18a bends in plane P2 and the second articulating joint 18b bends in a different plane P1 upon activation. Similar to that in the embodiments of FIGS. 2B and 3B, these articulating joints may also include through-holes and a cavity. These through-holes and cavity may be positioned such that the through-holes and cavity on the first articulating joint 18a aligns with through-holes and cavity on the second articulating joint 18b. These aligned through-holes enable the same elongate members to pass through and activate both the articulating joints (first and second articulating joints 18a, 18b). Planes P1 and P2 may be oriented with respect to each other in any manner. In some embodiments, planes P1 and P2 may be orthogonal to each other, while in other embodiments, they make a different angle between them.

Although FIG. 4 depicts a catheter 200 having two articulating joints each configured to bend in a different plane, this is only exemplary. Catheter 200 may, in general, include multiple sets of articulating joints arranged such that, each set of joints bends in a different bending plane. And, as discussed earlier, these joints may also be activated by different numbers of elongate members. Together, the articulating joints and elongate members of a catheter 200 may be configured to enable the catheter 200 to bend along different planes. For example, in one embodiment, a catheter may include three sets of articulating joints aligned to bend on bending planes that make a 120° angle with respect to each other. Three elongate members, each adapted to activate one set of articulating joints, may pass through the three articulating joints. In such an embodiment, the catheter may be configured to bend in the three planes upon activation. In some embodiments, the tubular section 20 between the first articulating joint 18a and second articulating joint 18b may be eliminated and the articulating joints may be directly attached to each other.

In some embodiments, stop mechanisms/features may also be incorporated in the articulating joints to control the range of motion of the joints. These stop mechanisms may include new components or features that are incorporated into parts of an articulating joint to limit the amount of deflection of the joint. For example, a projection 23 on one or both the arms of an articulating joint (or tubular section 20) may abut after a desired amount of deflection and act as a stop mechanism.

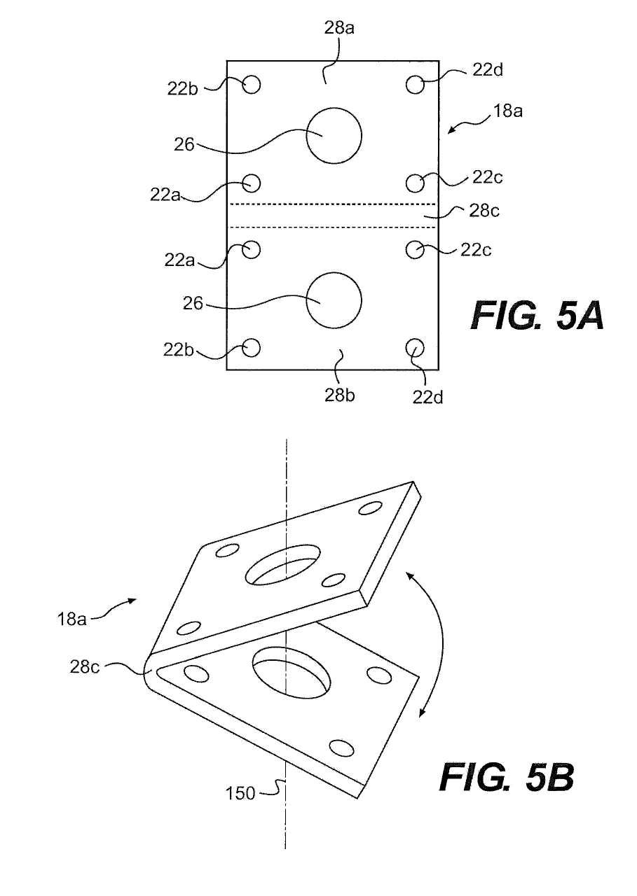

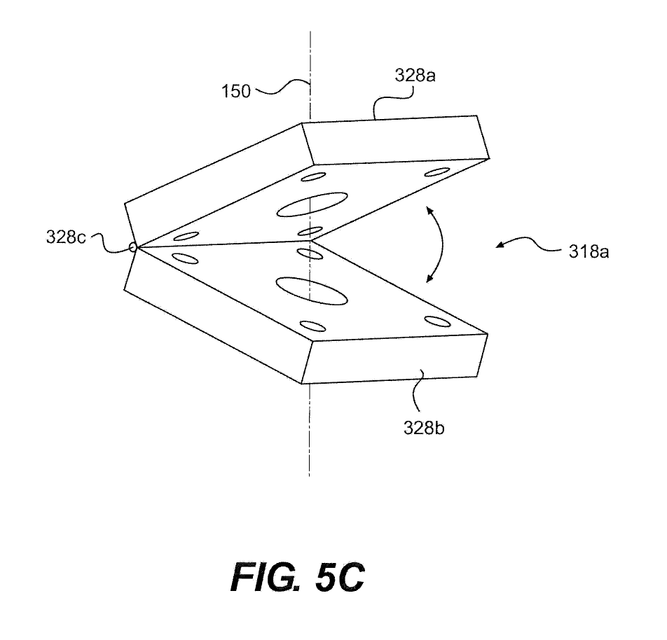

FIG. 5A is an illustration of a rectangular strip of material that may be bent to form an articulating joint discussed with reference to FIGS. 2A-2B, 3A-3B, and 4. Holes that form the through-holes 22a-22d and cavity 26 may be formed on the strip, and the strip folded at a bending section 28c to form a v-shaped articulating joint (such as, for instance the first articulating joint 18a of FIG. 5B). After folding, the first articulating joint 18a may include a first section 28a folded from a second section 28b at the bending section 28c. To activate the first articulating joint 18a, the free ends of the first articulating joint 18a may be pulled outwards and pressed inwards by a force applied by the elongate members coupled thereto. During activation, the first section 28a may rotate towards or away from the second section 28b at the bending section 28c. It should be noted that since bending occurs only about the bending section 28c, torque transmission about the longitudinal axis 150 remains unaffected. That is, applying a torque about the longitudinal axis 150 does not activate the first articulating joint 18a. Consequently, torque may be transmitted about the longitudinal axis 150 without rotating a section of the catheter on one side of the articulating joint relative to a section of the catheter on the opposite side of the articulating joint. In these embodiments, bending section 28c may act like a live hinge. Any material that allows for the formation of a live hinge between the first section 28a and second section 28b may be used to fabricate the articulating joint of these embodiments. Examples of such materials include stainless steel (302 series, type 17-4, etc.), spring steel, a shape memory alloy, plastics, etc.

In some embodiments, instead of a live hinge (that is, bending a strip of material to create a joint), two smaller strips 328a and 328b may coupled together by a pivot 328c to form a first articulating joint 318a as illustrated in FIG. 5C. Any type of pivot known in the art may be used as pivot 328c. In some embodiments, the pivot 328c may include a length of a material that passes through a hinge-like structure on strips 328a and 328b. The pivot 328c may enable the strips 328a and 328b to rotate relative to each other about the pivot 328c. These articulating joints may be arranged in any manner (such as, for example, as illustrated in FIGS. 2B and 3B) to form catheters that are configured to bend in multiple planes. In addition, pivot 328c may enable torque to be transmitted about the longitudinal axis 150 without relative rotation of the catheter on either sides of the articulating joint 318a. Although, in the preceding discussion, a strip of material having a rectangular shape is described as being used to create the articulating joints of FIGS. 5A and 5C, it should be noted that this is only exemplary and not a limitation. In general, material of any shape may be used to create an articulating joint. Some exemplary embodiments of such joints are described in the sections below.

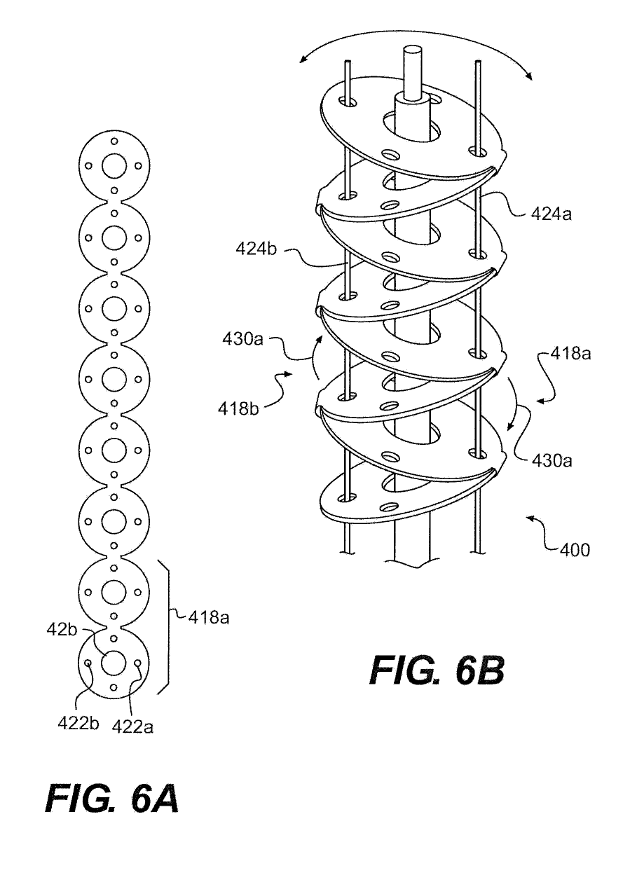

FIG. 6A illustrates an exemplary strip of material that may form a string of articulating joints (418a, 418b, etc.) of catheter 400 of FIG. 6B. A repeating pattern of connected substantially circular shapes as illustrated in FIG. 6A may be formed by stamping or another suitable manufacturing process. This starting material may be folded to form a repeating pattern of articulating joints 418a, 418b, etc. that alternate in the direction of bending. It is also contemplated that, as discussed with reference to FIG. 5C, instead of folding the starting material to form a joint, a pivot may also be used to form the joints. Two or more elongate members 424a and 424b may pass through through-holes 422a, 422b, etc. provided in the starting material to articulate the joints 418a and 418b. In the embodiment illustrated in FIG. 6B, pulling elongate member 424a will move the arms of articulating joints 418a and 418b in the direction of arrow 430a to bend catheter 400 in the clockwise direction. Similarly, pulling elongate member 424b will move the arms of the joints in the opposite direction and bend the catheter 400 in the counterclockwise direction. In some embodiments, a series or a string of articulating joints may extend substantially over the entire length of the catheter 400, while in other embodiments, a string of articulating joints may be provided in selected sections of catheter 400. Each of these strings may have the same length or have different lengths. A string of articulating joints of a different length may be formed by varying the number of joints in the string and/or by varying the dimensions of the joints. In the embodiment of FIG. 6B, each articulating joint is shown to have a simple bend between two substantially circular segments. However, other embodiments of articulating joints may have more complex bend shapes, such as, for example, a wave across an entire surface of a link. In some embodiments, the substantially circular segments that form the arms of the articulating joints (418a, 418b) of FIG. 6B may be replaced with other shapes, such as, for example, oval shapes, or other shapes that are optimized for bending. Although in the embodiment of catheter 400 of FIG. 6B, the articulating joints are arranged to bend in one bending plane, in other embodiments, these joints may be arranged to bend on different bending planes.

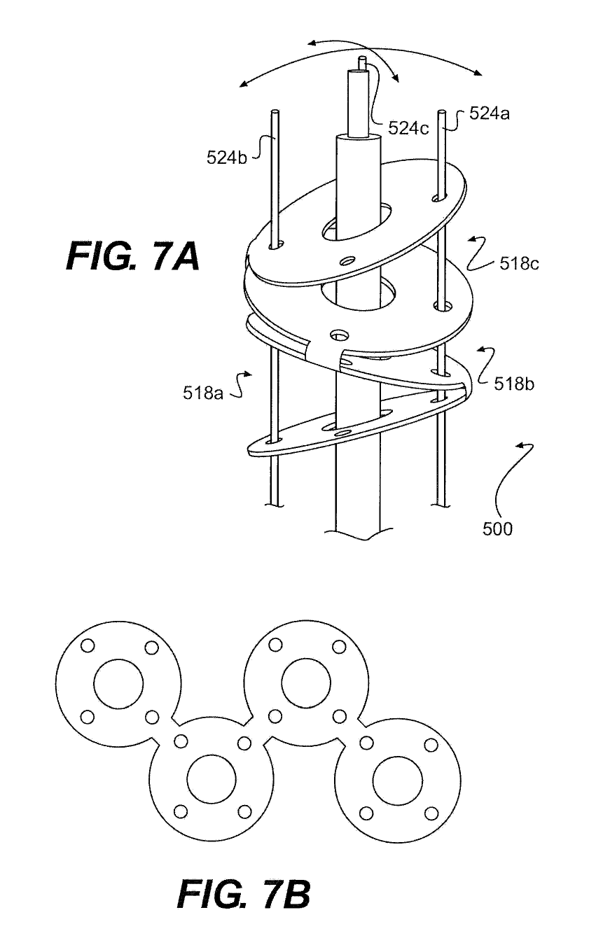

FIG. 7A shows an embodiment of a section of a catheter 500 which includes articulating joints arranged to bend in two different bending planes. FIG. 7B illustrates an exemplary starting material that may form the articulating joints 518a, 518b, and 518c of FIG. 7A. Any suitably shaped link that can be bend in different planes to form a string of articulating joints (as illustrated in FIG. 7B) may serve as the starting material. As in the embodiments discussed previously, these joints may have through-holes to pass elongate members (524a, 524b, 524c) that are adapted to articulate the joints. In the arrangement of joints illustrated in FIG. 7A, pulling elongate members 524a and 524b may bend catheter 500 in the plane of the paper and pulling elongate member 524c may bend the catheter out of the plane of the paper. These different planes may make any angle with respect to each other, such as, for example, 90° or any other angle. Although the articulating joints 518a and 518c that bend in different planes are illustrated as being positioned next to each other, in some embodiments, a string of joints that are configured to bend on one plane may be positioned together followed by a string of joints that are configured to bend on another plane. Although the embodiment of the catheter in FIG. 7A is only configured to bend in two planes, other embodiments of the catheter may include articulating joints that are configured to bend in more planes while allowing for torque transmission about the longitudinal axis without relative rotation of the catheter on either side of the articulating joints.

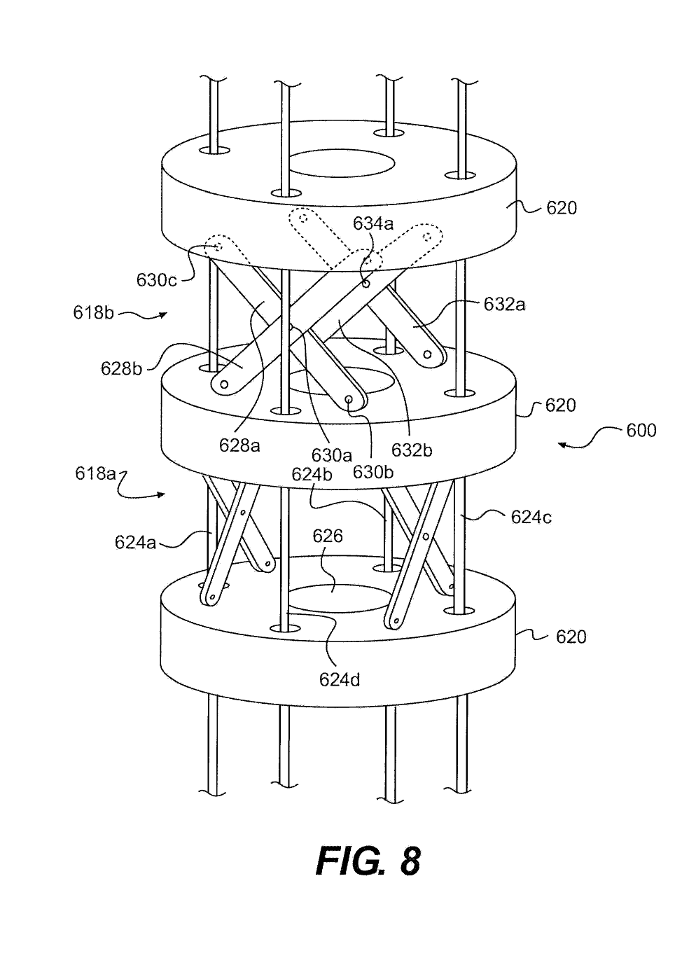

FIG. 8 illustrates another embodiment of a catheter 600 of the current disclosure. In this embodiment, articulating joints 618a, 618b may be formed by links that are coupled together at pivots. These pivoted links may couple tubular sections 620 of the catheter together, and may be configured to enable a section of the catheter 600 to rotate in a plane upon activation by an elongate member. For instance, second articulating joint 618b may be formed by links 628a and 628b that are coupled together at pivot 630a, and by links 632a and link 632b that are coupled together at pivot 634a. These links may also be coupled at either end to tubular sections 420 though pivots. For instance, link 628a may be coupled at either end to tubular sections 620 by pivots 630b and 630c. These links may be arranged to enable a cavity 626 to extend along the length of the catheter 600. In embodiments where cavity 626 extends through the middle of the catheter 600, these links may be positioned on either side of the cavity 626 in such a manner that the links stay within a spatial volume defined by the periphery of the cavity and an outer periphery of the tubular sections 620. When the links are thus positioned, these links will not interfere with a surgical tool or another device that extends into the body through the cavity 626, nor press against the walls of the body cavity through which catheter 600 passes. Through-holes may also extend through the tubular sections 620 to pass elongate members (624a, 624b, 624c, and 624d) therethrough. As described previously, these elongate members may be configured to activate the articulating joints 618a and 618b. Pulling of elongate members 624a and 624c may rotate articulating joint 618b about the pivots and bend a section of catheter 600 in a plane.

Catheter 600 may include multiple articulating joints that are positioned adjacent to each other along a length of the catheter. These multiple articulating joints may be configured to bend a length of the catheter 600 in a plane. These multiple articulating joints may have a similar structure as articulating joint 618b and may include links that are coupled together at pivots. In some embodiments, the links of these multiple articulating joints may arranged to bend different sections of the catheter along the same plane. While in other embodiments, the links of some of the articulating joints may be arranged to enable a section of the catheter to bend along a different plane. For instance, the catheter 600 may include a plurality of articulating joints 618a that are arranged to bend a section of the catheter in a first bending plane and a plurality of articulating joints 618b that are arranged to bend a different section of the catheter in a second bending plane. Pulling elongate members 624b and 624d may activate articulating joint 618a and bend a section of the catheter 600 in the first bending plane, and pulling elongate members 624a and 624c may activate articulating joint 618b and bend a section of the catheter 600 in the second bending plane. Although FIG. 8 illustrates only two articulating joints each configured to bend the catheter along a different plane, this is only exemplary. As described earlier, catheter 600 may include a string of articulating joints that are arranged to enable different sections of the catheter to bend in the same or different planes while allowing for torque transmission about the longitudinal axis. Several modifications may be made to articulating joints illustrated in FIG. 8. For instance, in some embodiments, the tubular sections 620 may be eliminated and/or the shapes of the links modified. In some embodiments, stops or other features may be incorporated into the articulating joints to limit the bending of the joints.

In use, a distal end of a catheter may be inserted into an opening (natural opening or an incision) of the body, and the catheter may be pushed into the body through a body tract. As the catheter travels through the body tract, the activation mechanism may be manipulated to controllably bend sections of the catheter in one or more different planes. Manipulating the activation mechanism may pull/push one or more elongate members coupled to the articulating joints to bend different sections of the catheter in the one or more bending planes. Different sections of the catheter may be bent in a plane to assist with the endoscopic procedure the catheter is used in. For instance, in some applications, the catheter may be bent so that the curvature of the catheter resembles the curvature of the body tract to reduce the trauma to the walls of the body tract. In some other applications, the sections of the catheter may be bent in different planes to enable the catheter to pass through an area of constriction in the body tract. After the distal end of the catheter is positioned at a desired location in the body, a torque (about the axis of the catheter) may be applied to the catheter at the proximal end to enable a surgical device coupled to the distal end to apply a desired force to an area of interest within the body. The arrangement of the articulating joints in the catheter may allow for torque transmission about the longitudinal axis without relative rotation of the catheter on either side of the articulating joints.

The embodiments described herein are exemplary only, and it will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only, with a true scope of the invention being indicated by the following claims.

What is claimed is:

1. An endoscopic device, comprising:
an elongate section extending from a proximal end to a distal end along a longitudinal axis, the elongate section being configured to transmit a desired torque about the longitudinal axis without relative rotation between the proximal end and the distal end;
a plurality of bend elements positioned on the elongate section, the plurality of bend elements including,
a first articulating joint configured to bend the elongate section in a first bending plane, and
a second articulating joint configured to bend the elongate section in a second bending plane different from the first bending plane.

2. The device of claim 1, wherein the first bending plane is orthogonal to the second bending plane.

3. The device of claim 1, wherein at least one of the first articulating joint and the second articulating joint includes a live hinge.

4. The device of claim 1, wherein the first articulating joint includes a stop mechanism that is configured to limit the amount of deflection of the first articulating joint.

5. The catheter of claim 1, wherein at least one of the first articulating joint and the second articulating joint includes a rectangular strip that is folded along a width of the strip.

6. The catheter of claim 1, wherein the first articulating joint and the second articulating joint are substantially similar parts that are oriented such that the first articulating joint bends in the first bending plane and the second articulating joint bends in the second bending plane.

7. The catheter of claim 1, wherein at least one of the first articulating joint and the second articulating joint includes one of a stainless steel, spring steel, a shape memory alloy, and a plastic material.

8. The catheter of claim 1, wherein the bend elements further include a third articulating joint that is configured to bend the elongate section in a third bending plane that is different from the first and second bending planes.

9. The catheter of claim 1, further including a string of first articulating joints in one section of the elongate section and a string of second articulating joints in a different section of the elongate section.

10. The catheter of claim 1, further including a first elongate member that is coupled to the first articulating joint and configured to bend the first articulating joint in the first bending plane.

11. The catheter of claim 10, further including a second elongate member that is coupled to the second articulating joint and configured to bend the second articulating joint in the second bending plane.

12. An endoscopic device, comprising:
an elongate section extending from a proximal end to a distal end along a longitudinal axis, the elongate section being configured to transmit a desired torque about the longitudinal axis without relative rotation between the proximal end and the distal end;
a lumen extending through the elongate section from the proximal end to the distal end; and
a plurality of first bend elements positioned along the elongate section and a plurality of second bend elements positioned along the elongate section, each bend element of the plurality of first bend elements being configured to bend the elongate section along a first bending plane and each bend element of the plurality of second bend elements being configured to bend the elongate section along a second bending plane different from the first bending plane.

13. The catheter of claim 12, wherein the first bending plane is orthogonal to the second bending plane.

14. The catheter of claim 12, wherein the plurality of first bend elements includes multiple first bend elements positioned proximate each other in a first length of the elongate section, and the plurality of second bend elements include multiple second bend elements positioned proximate each other in a second length of the elongate section, the first length being different from the second length.

15. The catheter of claim 12, wherein the plurality of first bend elements includes a strip of material that is folded to form multiple first bend elements that are each configured to bend in the first bending plane.

16. The catheter of claim 15, wherein the plurality of second bend elements includes multiple second bend elements that are folded from the strip of material and are each configured to bend in the second bending plane.

17. The catheter of claim 15, wherein at least one of the plurality of first bend elements and the plurality of second bend elements includes a pivot.

18. A method of using an endoscopic catheter, comprising:
inserting a distal end of the catheter into an opening in ct of a body, the catheter including an elongate section extending along a longitudinal axis and a plurality of bend elements, the elongate section being configured to transmit a torque about the longitudinal axis without relative rotation between regions of the elongate section on either side of the plurality of bend elements, the plurality of bend elements including at least a first and a second articulating joint, the first and the second articulating joints being configured to bend the elongate section in different bending planes;
activating the first articulating joint to bend the first articulating joint in a first bending plane; and
activating the second articulating joint to bend the second articulating joint in the second bending plane.

19. The method of claim 18, wherein activating the first articulating joint includes bending the first articulating joint in the first bending plane without bending the second articulating joint.

20. The method of claim 18, further including applying a torsional force at a proximal end of the catheter to transmit the force to a distal end of the catheter without causing relative rotation between the proximal and distal ends of the catheter.
